(19) United States
(12) Patent Application Publication
(10) Pub. No.: US 2018/0028150 A1
(43) Pub. Date: Feb. 1, 2018

(54) ULTRASONIC PROBE AND SUBJECT INFORMATION ACQUISITION APPARATUS

(71) Applicant: CANON KABUSHIKI KAISHA, Tokyo (JP)

(72) Inventor: Atsushi Kandori, Ebina-shi (JP)

(21) Appl. No.: 15/660,787

(22) Filed: Jul. 26, 2017

(30) Foreign Application Priority Data
Jul. 29, 2016 (JP) 2016-150108

(51) Int. Cl. A61B 8/00; G01N 29/06; B06B 1/06

(52) U.S. Cl. CPC A61B 8/4281; B06B 1/0622; G01N 29/0618; H05K 3/36

(57) ABSTRACT

An ultrasonic probe includes an oscillator including a pair of electrodes, wherein the ultrasonic probe is configured to transmit and receive an ultrasonic wave by an oscillation of the oscillator, and wherein a wiring for transmitting a transmission signal for transmitting the ultrasonic wave by the oscillator is connected to one of the pair of electrodes, and a preamplifier configured to amplify a reception signal acquired from the reception of the ultrasonic wave by the oscillator is connected to the other of the pair of electrodes.

### BACKGROUND

### Field of the Disclosure

The present disclosure relates to an ultrasonic probe capable of transmitting and receiving an ultrasonic wave and a subject information acquisition apparatus using this ultrasonic probe.

### Description of the Related Art

There is known a method that images an inside of a subject with use of an ultrasonic imaging technique that acquires information inside the subject by transmitting an ultrasonic wave to the subject and receiving the ultrasonic wave reflected from inside the subject. An apparatus for carrying out the ultrasonic imaging includes a signal processing unit that processes transmission and reception signals of the ultrasonic wave and an ultrasonic probe connected to the signal processing unit via a cable. An ultrasonic oscillator (hereinafter may be simply abbreviated as an oscillator) provided to the ultrasonic probe is used to transmit and receive the ultrasonic wave. The transmission signal for transmitting the ultrasonic wave is generated by the signal processing unit and is transmitted to the oscillator via the cable. Further, the reception signal generated from the reception of the ultrasonic wave by the oscillator is transmitted to the signal processing unit via the cable and is converted into a digital signal.

Japanese Patent Application Laid-Open No. 6-217980 discusses an ultrasonic diagnostic apparatus in which some signal transmission paths from the signal processing unit to the probe are shared between the transmission signal and the reception signal of the ultrasonic wave.

In the ultrasonic diagnostic apparatus discussed in Japanese Patent Application Laid-Open No. 6-217980, a preamplifier for amplifying the reception signal of the ultrasonic wave is provided in the path for transmitting the transmission and reception signals of the ultrasonic wave. Also, Japanese Patent Application Laid-Open No. 6-217980 discusses a configuration in which protection circuits for protecting the preamplifier from a high voltage applied to transmit the ultrasonic wave are provided in front of and at the back of the preamplifier.

The present inventor(s) has(have) identified various issues with the ultrasonic diagnostic apparatus discussed in Japanese Patent Application Laid-Open No. 6-217980. For example, since the protection circuits for protecting the preamplifier should be resistant to the high voltage, the protection circuits have increase in a parasitic capacitance, a wiring resistance, and the like. This may lead to deterioration of a reception characteristic of the preamplifier.

### SUMMARY

According to an aspect of the present disclosure, an ultrasonic probe including an oscillator including a pair of electrodes, wherein the ultrasonic probe is configured to transmit and receive an ultrasonic wave by an oscillation of the oscillator, and wherein a wiring for transmitting a transmission signal for transmitting the ultrasonic wave by the oscillator is connected to one of the pair of electrodes, and a preamplifier configured to amplify a reception signal acquired from the reception of the ultrasonic wave by the oscillator is connected to the other of the pair of electrodes.

Further features of the present disclosure will become apparent from the following description of exemplary embodiments with reference to the attached drawings.

### BRIEF DESCRIPTION OF THE DRAWINGS

FIGS. 1A to 1C each illustrate an ultrasonic probe according to a first exemplary embodiment of the present disclosure.

FIGS. 2A to 2C each illustrate an ultrasonic probe according to a second exemplary embodiment of the present disclosure.

FIG. 3 illustrates an ultrasonic probe according to a third exemplary embodiment of the present disclosure.

FIG. 4 illustrates an ultrasonic probe according to a fourth exemplary embodiment of the present disclosure.

FIG. 5 illustrates an ultrasonic probe according to a fifth exemplary embodiment of the present disclosure.

FIG. 6A illustrates an ultrasonic probe according to a sixth exemplary embodiment of the present disclosure.

FIG. 6B illustrates an ultrasonic probe according to another example of the sixth exemplary embodiment of the present disclosure.

FIG. 7 illustrates an ultrasonic probe according to a seventh exemplary embodiment of the present disclosure.

FIG. 8 illustrates an ultrasonic probe according to an eighth exemplary embodiment of the present disclosure.

FIG. 9 illustrates a subject information acquisition apparatus according to a ninth exemplary embodiment of the present disclosure.

FIG. 10 illustrates a subject information acquisition apparatus according to a tenth exemplary embodiment of the present disclosure.

FIGS. 11A to 11D each illustrate an example of a diagram of a circuit included in the ultrasonic probe according to each of the exemplary embodiments of the present disclosure.

FIG. 12 illustrates an ultrasonic probe including a built-in preamplifier.

### DESCRIPTION OF THE EMBODIMENTS

In the following description, exemplary embodiments of the present disclosure will be described with reference to the drawings. The present exemplary embodiments are configured to prevent a high-voltage pulse signal for transmitting an ultrasonic wave from being applied to a preamplifier configured to amplify a reception signal of the ultrasonic wave, thereby eliminating the necessity of a protection circuit for the preamplifier. To achieve that, the present exemplary embodiments are configured in such a manner that the preamplifier configured to amplify the reception signal of the ultrasonic wave and a wiring used to transmit the transmission signal of the ultrasonic wave are connected to different electrodes of a pair of electrodes included in an oscillator. Since no protection circuit for the preamplifier is provided, this configuration can prevent deterioration of a reception characteristic of the preamplifier due to the protection circuit.

In the following description, details thereof will be described with use of specific configurations.

An ultrasonic probe according to a first exemplary embodiment has the following characteristics. The pair of opposite electrodes included in the oscillator that transmits and receives the ultrasonic wave is separated for each element unit that transmits and receives the ultrasonic wave. Further, the wiring used to apply the transmission signal of the ultrasonic wave is connected to one of the pair of opposite electrodes, and the preamplifier (a detection circuit) configured to detect the reception signal of the ultrasonic wave is connected to the other of the pair of opposite electrodes.

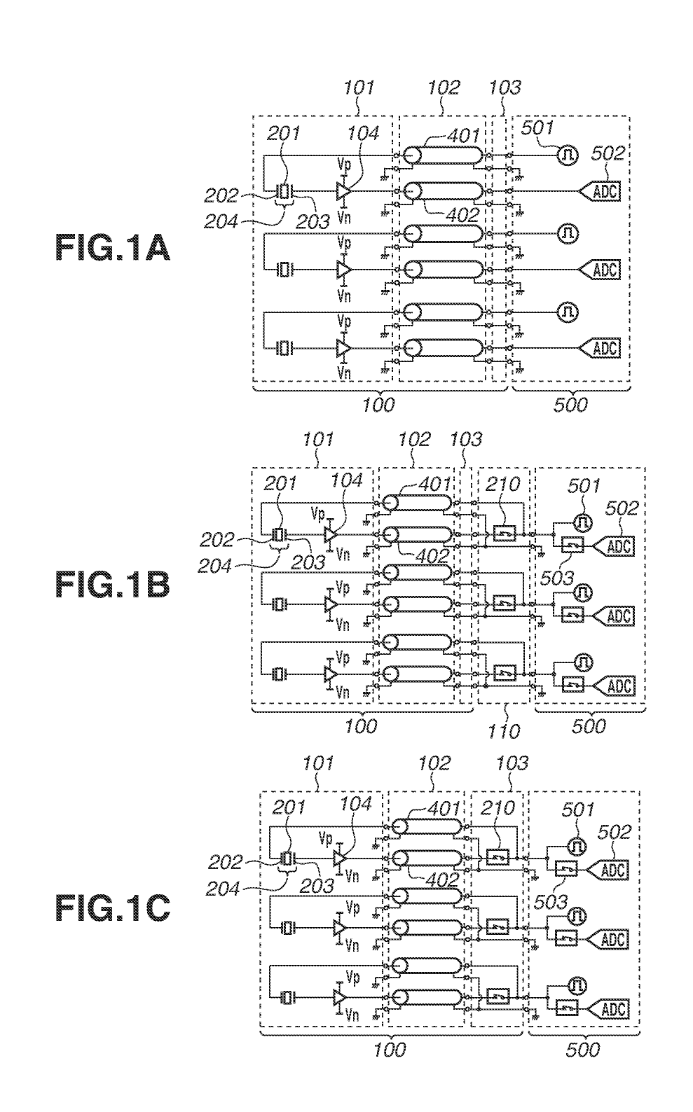

The present exemplary embodiment will be described in detail with reference to FIGS. 1A to 1C. FIG. 1A is a schematic diagram for illustrating a circuit configuration of the ultrasonic probe and a signal processing unit according to the present exemplary embodiment. FIG. 1A illustrates an ultrasonic probe 100, a probe casing 101, a cable 102, a connector 103, and a preamplifier 104. Further, FIG. 1A illustrates a piezoelectric element 201, and a first electrode 202 and a second electrode 203, which are the one and the other of the pair of electrodes, respectively. An ultrasonic oscillator (hereinafter may be abbreviated as an oscillator) 204 includes the piezoelectric element 201, the first electrode 202, and the second electrode 203. The oscillator using the piezoelectric element can also be restated as a piezoelectric (ultrasonic) transducer.

A signal processing unit 500 processes the transmission signal and the reception signal of the ultrasonic wave, and a voltage pulse generation unit 501 generates a high-voltage pulse. An analog-digital conversion unit (AD converter) 502 converts an analog signal into a digital signal.

The ultrasonic probe 100 includes the probe casing 101, the cable 102, and the connector 103. A plurality of oscillators 204, each of which is the element that transmits and receives the ultrasonic wave, is provided in the probe casing 101, and is electrically connected to the connector 103 via the cable 102.

The connector 103 can be attached to the signal processing unit 500, and the high-voltage pulse generated by the voltage pulse generation unit 501 in the signal processing unit 500 can be transmitted to the piezoelectric element 201 via the connector 103. Further, the preamplifier 104 amplifies the reception signal generated from the reception of the ultrasonic wave by the oscillator 204, and this amplified signal can be transmitted to the analog-digital conversion unit 502 in the signal processing unit 500 via the connector 103. A plurality of cables is further provided in the cable 102, which means that the cable 102 employs a configuration that is robust against an exogenous noise, is highly flexible, and is constructed by bundling micro coaxial cables together.

Each of the oscillators 204 includes the first electrode 202 and the second electrode 203 opposite from each other. The first electrode 202 and the second electrode 203 are electrically separated for each unit by which the oscillator 204 transmits and receives the ultrasonic wave, in other words, for each element unit for the transmission/reception wave. In FIG. 1A, the oscillators 204 are electrically separated one by one, but, in a case where a plurality of oscillators forms a single element that transmits and receives the ultrasonic wave, the plurality of oscillators forming the single element may be electrically connected to each other or one another. In this case, the oscillators belonging to different elements are electrically separated from each other or one another.

The ultrasonic probe 100 according to the present exemplary embodiment is configured to be able to transmit and receive the ultrasonic wave using a piezoelectric effect of the piezoelectric element 201 provided between the pair of electrodes (between the electrodes 202 and 203).

FIG. 1A illustrates a configuration including three piezoelectric elements 201, but the present exemplary embodiment may be a configuration including two or less piezoelectric element(s), or four or more piezoelectric elements. The same also applies to the exemplary embodiments that will be described below.

The second electrode 203 of the oscillator 204 is connected to an input terminal of the preamplifier 104, and is connected to a terminal in the connector 103 via a reception cable 402 in the cable 102. This terminal in the connector 103 is connected to an input terminal of the analog-digital conversion unit 502 in the signal processing unit 500. In the present disclosure, unless otherwise specifically indicated, being connected means being electrically connected and includes even such a connection that objects are connected to each other with another object interposed therebetween.

On the other hand, the first electrode 202 of the oscillator 204 is connected to a terminal in the connector 103 via a transmission cable 401 in the cable 102. This terminal in the connector 103 is connected to an output terminal of the voltage pulse generation unit 501 in the signal processing unit 500.

According to the configuration of the present exemplary embodiment, a path used to transmit the high-voltage pulse when the ultrasonic wave is transmitted, and a path used to amplify the reception signal when the ultrasonic wave is received are separated from each other. More specifically, the second electrode 203 of the oscillator 204 is used as the electrode to which the preamplifier 104 configured to amplify the reception signal of the ultrasonic wave is connected, and the first electrode 202 is used as the electrode to which the path of the transmission signal based on the high-voltage pulse is connected. Due to this configuration, no protection circuit for protecting the preamplifier 104 from the high voltage is connected to the input side of the preamplifier 104. Therefore, this configuration can prevent deterioration of a reception characteristic of the preamplifier 104 that is caused by, for example, generation of a parasitic capacitance generated due to the utilization of the protection circuit. Therefore, according to the ultrasonic probe 100 of the present exemplary embodiment, the reception characteristic of the preamplifier 104 is less likely deteriorated, and therefore an excellent reception characteristic can be acquired.

In the present disclosure, the preamplifier refers to an amplification unit (an amplifier) playing a role of amplifying the signal of the ultrasonic wave received by the ultrasonic probe in advance before this signal is transmitted to an amplifier (not illustrated) provided to an ultrasonic apparatus including the signal processing unit.

### First Exemplary Modification of First Exemplary Embodiment

In the present exemplary embodiment, the signal processing unit 500 has been described as being configured to include the terminal for transmitting the transmission signal of the ultrasonic wave and the terminal for transmitting the reception signal independently of each other, but the present disclosure is not limited thereto. For example, as illustrated in FIG. 1B, the present exemplary embodiment may include a transmission/reception (T/R) switch (a high voltage block unit) 210 that selectively connects the terminal for transmitting the ultrasonic wave and the terminal for receiving the ultrasonic wave to the ultrasonic probe 100. Then, the present exemplary embodiment may be configured in such a manner that the T/R switch 210 is disposed at a portion where the cable 102 of the ultrasonic probe 100 and the signal processing unit 500 are connected to each other as an adapter 110. In this case, the T/R switch 210 is a switch having a function of establishing conductivity between input and output terminals when a low voltage (normally, several volts or lower) is applied to the terminal, and blocking the conductivity between the input and output terminals when a high voltage is applied to the terminal. In other words, the wiring used for transmitting the transmission signal and the wiring used for transmitting the reception signal form a common wiring in which they partially share a same wiring therebetween. This common wiring is provided with the switch configured to switch a state capable of transmitting the transmission signal and a state capable of transmitting the reception signal.

The configuration illustrated in FIG. 1B includes the T/R switch 210. Therefore, when the high-voltage pulse is applied at the time of the transmission of the ultrasonic wave, the path of the wiring connected to the preamplifier 104 is closed off, and the high voltage is prevented from being applied to the output terminal of the preamplifier 104. Further, at the time of the reception of the ultrasonic wave, because the output signal output from the preamplifier 104 is approximately several hundred microvolts, the T/R switch 210 does not block the conductivity and therefore the reception signal is transmitted to the signal processing unit 500 without being blocked. In the present exemplary embodiment, the T/R switch 210 is connected to the output terminal side of the preamplifier 104. Thus, the reception characteristic of the preamplifier 104 is hardly affected. If the T/R switch 210 is not provided, the high-voltage pulse at the time of the transmission is applied to the output terminal of the preamplifier 104, and the preamplifier 104 may be broken in the worst case. The configuration according to the present exemplary embodiment can prevent that. In the configuration illustrated in FIG. 1B, the high voltage is not applied to the preamplifier 104, and therefore the terminal to be used for both the transmission and the reception can be also used as a terminal for use in the transmission and the reception that is connected to the apparatus side. Generally, a terminal connecting the ultrasonic imaging apparatus and the ultrasonic probe to each other is configured in such a manner that the same terminal is used for both the transmission and the reception. Then, according to the present configuration, the reception characteristic of the preamplifier 104 is less likely deteriorated, and, in addition to that, the same terminal can be used for both the transmission and the reception.

This configuration enables the ultrasonic probe 100 according to the present exemplary embodiment to be connected without changing a configuration of a conventional ultrasonic imaging apparatus. Therefore, an ultrasonic probe according to a conventional configuration and the ultrasonic probe 100 according to the present exemplary embodiment can be used together and employed for the same ultrasonic imaging apparatus.

### Second Exemplary Modification of First Exemplary Embodiment

Further, as illustrated in FIG. 1C, the adapter 110 illustrated in FIG. 1B can also be configured to be built in the connector 103 when the cable 102 and the signal processing unit 500 are connected to each other. This configuration can limit members required to be detached and attached when the ultrasonic probe 100 according to the present exemplary embodiment is replaced to only the connector 103. As a result, the ultrasonic probe 100 can be replaced with similar labor and work to the conventional ultrasonic probe. The ultrasonic probe 100 according to the present exemplary embodiment that is configured as illustrated in FIG. 1C can be therefore used with similar operability to the conventional ultrasonic probe.

### Comparative Example

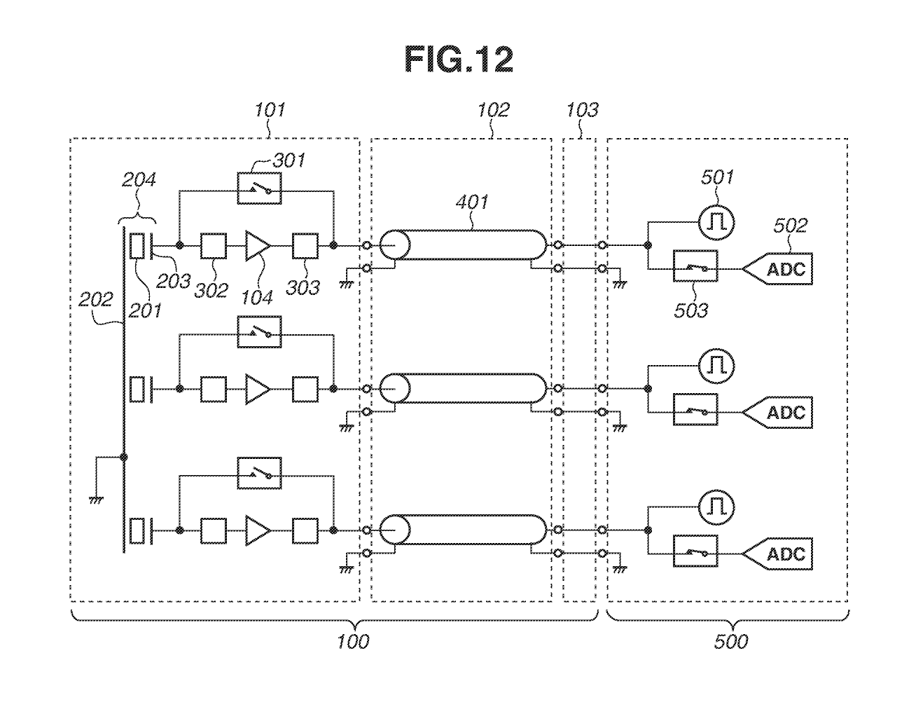

As illustrated in FIG. 12, the ultrasonic probe 100 is sometimes constructed with use of such a configuration that the preamplifier (the detection circuit) 104 configured to amplify the reception signal generated from the ultrasonic wave is built in the ultrasonic probe 100 to improve the reception characteristic of the ultrasonic probe 100. In this case, since the cable 102 connecting the signal processing unit 500 and the ultrasonic probe 100 to each other is used for both the transmission and the reception, protection circuits 302 and 303 should be disposed in front of and at the back of the preamplifier 104 (the input and output terminal sides) to prevent the preamplifier 104 from being broken due to the high-voltage pulse for the transmission. Further, a diode 301 should be disposed to prevent the reception signal from being superimposed as the transmission signal. Since the diode 301 and the protection circuits 302 and 303 should be resistant to the high voltage, each of the circuits have increases in a parasitic capacitance, a wiring resistance, and the like. This may lead deterioration of the reception characteristic of the preamplifier 104 compared to the characteristic of the preamplifier 104 disposed alone.

A second exemplary embodiment is different from the first exemplary embodiment in terms of an oscillator, i.e., a capacitive transducer is used as an oscillator, and is similar to the first exemplary embodiment except for that unless otherwise specifically described.

Hereinafter, the capacitive transducer (a capacitive micro-machined ultrasonic transducer) will be abbreviated as a CMUT.

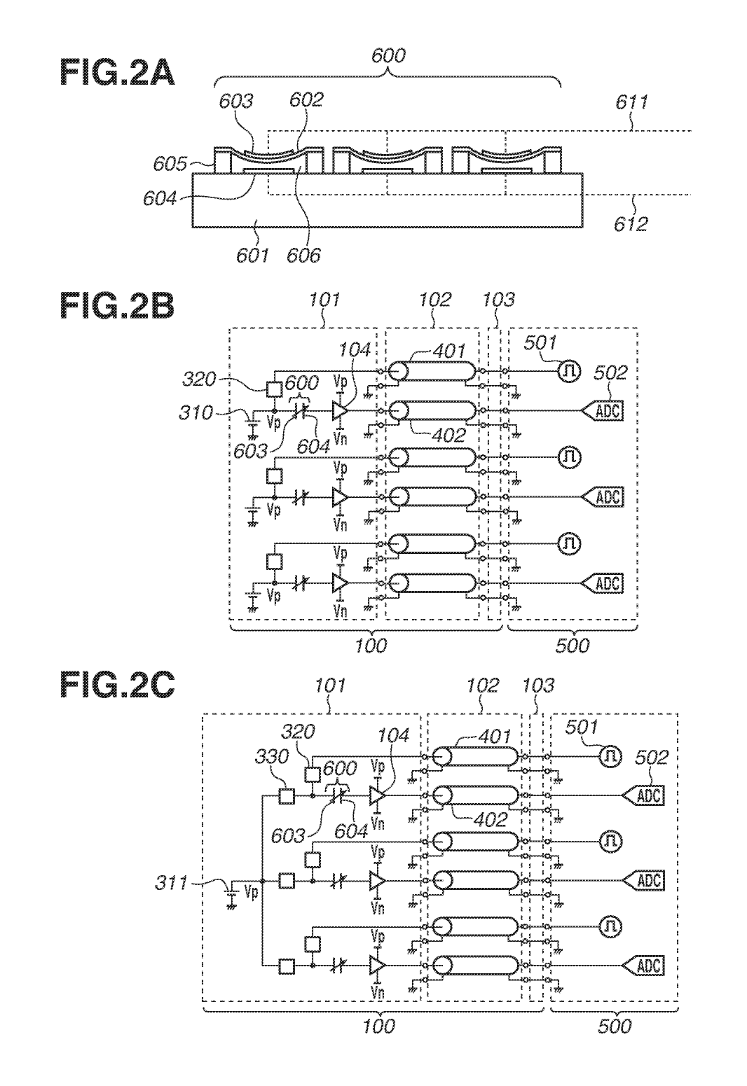

FIG. 2A is a schematic diagram of a cross section illustrating a structure of an example of the CMUT, and FIG. 2B is a schematic diagram illustrating a circuit included in the ultrasonic probe 100 according to the present exemplary embodiment. FIG. 2A illustrates a CMUT 600, a chip (a substrate) 601, a membrane 602, a first electrode 603, a second electrode 604, and a space 606. The CMUT 600 transmits and receives the ultrasonic wave with use of an oscillation membrane including the membrane 602 and the first electrode 603. The oscillation membrane is supported by a support portion 605. Further, a first wiring 611 is connected to the first electrode 603, and a second wiring 612 is connected to the second electrode 604.

The CMUT 600 is fabricated on the silicon chip 601 using the microelectromechanical systems (MEMS) process, which is an application of the semiconductor process. The CMUT 600 is characterized by its capability to increase a range of a reception frequency compared to the piezoelectric ultrasonic transducer.

A cross-sectional configuration of the CMUT 600 will be described. The membrane 602 is supported on the chip 601 by the support portion 605, and is configured to oscillate by receiving an acoustic wave (the ultrasonic wave). The first electrode 603 is disposed on the membrane 602, and the second electrode 604 is disposed at a position on the chip 601 opposite from the first electrode 603. The first electrode 603 and the second electrode 604 located opposite of the membrane 602 and the space 606 from each other will be referred to as a cell as one pair.

The first electrode 603 is extended out of the chip 601 via the first wiring 611, and is connected to a direct-current voltage generation unit (not illustrated) beyond that. A potential difference of several dozen volts to several hundred volts is generated between the first electrode 603 and the second electrode 604 due to the direct-current voltage generation unit.

The second electrode 604 is extended out of the chip 601 via the second wiring 612, and is connected to the preamplifier 104 (not illustrated) beyond that. When the membrane 602 and the first electrode 603 oscillate, a distance between the first electrode 603 and the second electrode 604 changes. As a result, an electrostatic capacitance between these electrodes 603 and 604 changes. Since there is the potential difference between the electrodes 603 and 604, an extremely low current occurs in correspondence with the change in the capacitance. The extremely low current is amplified by the preamplifier 104 connected beyond the second electrode 604, and is converted from the current into a voltage.

The CMUT 600 should be equipped with this direct-current voltage generation unit, and therefore cannot be directly applied to the configuration of the first exemplary embodiment. In the present disclosure, special configurations for using the CMUT including the direct-current voltage generation unit will be each described in the following description.

As illustrated in FIG. 2A, a plurality of cells is disposed on the chip 601. The first electrodes 603 in cells located in a region having a predetermined area are electrically connected to each other or one another, and, similarly, the second electrodes 604 in the cells in the region having the predetermined area are also electrically connected to each other or one another. This region where the first electrodes 603 and the second electrodes 604 are connected to each other or one another serves as the element unit (referred to as the element) by which the CMUT 600 transmits and receives the ultrasonic wave. The ultrasonic probe 100 according to the present exemplary embodiment includes preamplifiers 104 as many as the transmission and reception elements, and is configured to output the reception signal of the ultrasonic wave for each of the individual elements. Further, when transmitting the ultrasonic wave, the ultrasonic probe 100 according to the present exemplary embodiment can also independently apply the transmission pulse to the electrode element by element, and therefore can transmit the ultrasonic wave element by element. An example of a size of the cell is from several hundred micrometers to several millimeters, and an example of the number of cells in one element is from one hundred cells to several thousand cells.

The ultrasonic probe 100 according to the present exemplary embodiment is configured to be able to transmit and receive the ultrasonic wave using the change in the distance between the pair of electrodes due to the oscillation of the oscillator including the space 606 between the pair of electrodes (603 and 604).

In the present exemplary embodiment, the CMUT 600 is used instead of the oscillator 204, so that the ultrasonic probe 100 can cover a wide range of the reception frequency of the ultrasonic wave and detect various frequency components of the ultrasonic wave derived from the subject. This enables further more information regarding the subject to be acquired.

In FIG. 2A, the ultrasonic probe 100 has been described as being configured in such a manner that the first electrode 603 is disposed on the oscillation membrane 602 and the second electrode 604 is disposed on the chip 601, but the present exemplary embodiment is not necessarily limited thereto. A reverse combination can also be used in a similar manner. Further, in FIG. 2A, the ultrasonic probe 100 has been described with use of the configuration in which the direct-current voltage generation unit is connected to the first electrode 603 and the preamplifier 104 is connected to the second electrode 604, but the present exemplary embodiment is not necessarily limited thereto. A reverse combination can also be used in a similar manner.

As described above, the CMUT 600 is used with the potential difference of several dozen volts to several hundred volts (a bias voltage Vb) generated between the first electrode 603 and the second electrode 604. A configuration of the ultrasonic probe 100 according to the present exemplary embodiment in the case where the CMUT 600 is used as the oscillator will be described with reference to FIG. 2B. FIG. 2B illustrates a direct-current potential generation unit (a direct-current voltage application unit) 310 and a voltage level shift unit (a transmission signal level shift unit) 320.

The configuration in the case of the use of the CMUT 600 as the oscillator is different from the first exemplary embodiment in terms of the direct-current potential generation unit 310 which is provided independently and connected to the first electrode 603 of each CMUT 600. The input terminal of the preamplifier (the detection circuit) 104 for amplifying the reception signal is connected to the second electrode 604. A positive power source Vp and a negative power source Vn are connected to the preamplifier (the detection circuit) 104 based on a ground potential. When the ultrasonic wave is not received, the input terminal of the preamplifier (the detection terminal) 104 is set to a reference potential that the preamplifier 104 has. The reference potential is normally the ground potential at a middle point between the positive power source Vp and the negative power source Vn. Because a direct-current voltage is applied to the first electrode 603 while the other second electrode 604 is set to the reference potential that the preamplifier 104 has, a direct-current potential difference (a voltage) can be generated between the opposite electrodes 603 and 604.

The CMUT 600 may have a variation in the characteristic thereof due to a variation in manufacturing, and may have a variation in the transmission/reception characteristic of the oscillation membrane 602. The configuration illustrated in FIG. 2B can apply a different potential to each of the CMUTs 600, and therefore can adjust the potential difference to an optimum potential difference by correcting the variation in the transmission/reception characteristic of the oscillation membrane 602. Consequently, the CMUT 600 keeping a further even transmission/reception characteristic can be provided. Further, even if one element is broken and a predetermined potential difference becomes unable to be applied, the direct-current potential generation unit 310 provided independently allows the other elements to remain usable without being affected thereby.

Further, the ultrasonic probe 100 according to the present exemplary embodiment is characterized by the utilization of the voltage level shift unit (the transmission signal level shift unit) 320 provided between the one of the electrodes (the first electrode 603) and the transmission cable 401 used to transmit the transmission voltage. The voltage level shift unit (the transmission signal level shift unit) 320 has a function of being able to add a voltage corresponding to a reference to a signal based on a low voltage by a magnitude of the high voltage Vb (a voltage shift).

Normally, the high-voltage pulse generated by the voltage pulse generation unit 501 in the control unit 500 (signal processing unit 500) is generated based on the ground potential. Meanwhile, since the first electrode 603 of the CMUT 600 is connected to the direct-current potential generation unit 310 (the potential generation unit), the first electrode 603 has a potential difference from the high-voltage pulse and the high-voltage pulse thus cannot be transmitted only by an electrical connection.

The present exemplary embodiment acquires an effect of allowing the CMUT 600 to appropriately operate by adding the configuration of the voltage level shift unit (the transmission signal level shift unit) 320 in correspondence with the direct-current potential generation unit 310, which should be included in the CMUT 600.

The ultrasonic probe 100 according to the present exemplary embodiment includes the voltage level shift unit (the transmission signal level shift unit) 320 between the first electrode 603 and the transmission cable 401 used to transmit the transmission voltage. Accordingly, the potential difference (the voltage) of the high-voltage pulse based on the ground potential can be shifted to the potential generated by the direct-current potential generation unit 310. As a result, the range of the reception frequency of the ultrasonic wave can be increased with respect to the ultrasonic probe 100 using the CMUT 600 as the oscillator. Similarly, the transmission of the ultrasonic wave can be driven by the control unit 500 of the conventional ultrasonic imaging.

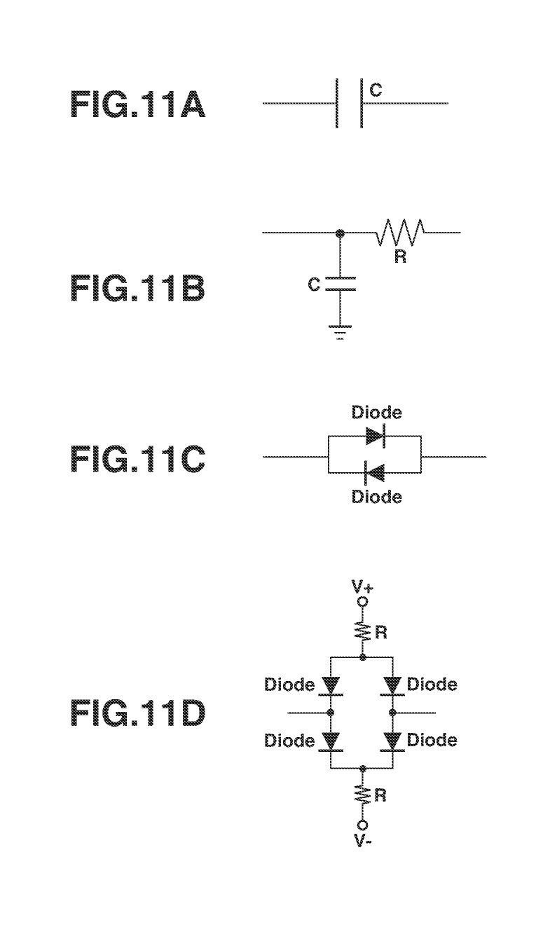

The voltage level shift unit (the transmission signal level shift unit) 320 may be any unit capable of converting the direct-current voltage level of the voltage (performing an operation of adding the bias voltage Vb) and allowing an alternating-current signal to be transmitted therethrough. For example, the voltage level shift unit 320 can be easily realized by using a capacitor as illustrated in FIG. 11A. Further, a voltage level shift circuit using a high-voltage transistor can also be used therefor.

According to the ultrasonic probe 100 of the present exemplary embodiment, it is possible to provide the ultrasonic probe including the built-in preamplifier that can acquire an excellent wideband reception characteristic.

In the present exemplary embodiment, the ultrasonic probe 100 has been described as being configured to include the direct-current voltage application unit 310 for each element unit of the CMUT 600 as illustrated in FIG. 2B, but the present disclosure is not limited to this configuration.

### Exemplary Modification of Second Exemplary Embodiment

As illustrated in FIG. 2C, an exemplary modification of the present exemplary embodiment is different from the previous example of the present exemplary embodiment in terms of utilization of a direct-current voltage generation unit 311 in common and an alternating- current voltage separation unit 330. In FIG. 2B, the ultrasonic probe 100 includes the direct-current voltage application unit 330 for each element unit of the CMUT 600, but, in FIG. 2C, the ultrasonic probe 100 includes the plurality of oscillators and the direct-current voltage generation unit 311 is connected to and shared between the plurality of oscillators 204. In FIG. 2C, the ultrasonic probe 100 includes the alternating-current voltage separation unit 330 between the direct-current voltage generation unit 311 in common and the first electrode 603 of each of the elements. The alternating-current voltage separation unit 330 has a characteristic of transmitting a direct-current voltage from the direct-current voltage generation unit 311 to the first electrode 603. On the other hand, the alternating-current voltage separation unit 330 has a characteristic of not transmitting, to the direct-current voltage generation unit 311 side, the high-voltage pulse for the transmission that is generated by the voltage pulse generation unit 501 in the control unit 500 and transmitted via the transmission cable 401 and the voltage level shift unit (the transmission signal level shift unit) 320. Due to these characteristics, even when the high-voltage pulse is applied to the first electrode 603 with a different magnitude or at a different timing for each of the transmission and reception elements, such a high-voltage pulse is not transmitted to the direct-current voltage generation unit 311 side. Consequently, even when the direct-current voltage generation unit 311 in common is used, the high-voltage pulse signal of a different element is not applied to another element. The present exemplary modification thus enables each of the elements to independently drive.

The alternating-current voltage separation unit 330 can be realized with use of any unit having a characteristic of permitting a direct-current voltage to be transmitted therethrough only in one direction and prohibiting an alternating-current voltage of a predetermined frequency or higher from being transmitted therethrough in an opposite direction. For example, as illustrated in FIG. 11B, the alternating-current voltage separation unit 330 can be realized by a simple filter configuration. The alternating-current voltage separation unit 330 according to the present exemplary embodiment can also be realized with use of not only the configuration illustrated in FIG. 11B but also another filter configuration in a similar manner as long as this configuration has the required function. Further, the alternating-current voltage separation unit 330 is configured in such a manner that, even when an abnormality has occurred in some element and the application of the direct-current potential is stopped, the other elements are not affected by the abnormality. More specifically, the alternating-current voltage separation unit 330 includes a resistor as illustrated in FIG. 11B, and therefore can make it difficult to change the direct-current potential to be applied to the other elements even when a resistance between the electrodes of the element significantly reduces. As a result, even if one element is broken and the predetermined potential difference becomes unable to be applied, the other elements can remain usable without being affected thereby despite that the direct-current potential generation unit 311 are in common among them.

According to the ultrasonic probe 100 of the other configuration of the present exemplary embodiment, it is possible to provide the ultrasonic probe including the built-in preamplifier that is compact because requiring only one direct-current voltage generation unit and has an excellent wideband reception characteristic.

In the present exemplary embodiment, the ultrasonic probe 100 has been described as being configured in such a manner that only the first electrode 603 is disposed on the oscillation membrane 602, but the present exemplary embodiment is not limited thereto. The present exemplary embodiment can also be applied in a similar manner to, for example, a configuration in which an insulating membrane is further disposed on the first electrode 603 on the oscillation membrane 602.

In the present exemplary embodiment, the ultrasonic probe 100 has been described as being configured in such a manner that the preamplifier (the detection circuit) 104 supplies both the power sources (Vp and Vn), but the present exemplary embodiment is not limited thereto. For example, the ultrasonic probe 100 may be configured to supply a voltage generated by the single power source Vp to the CMUT 600.

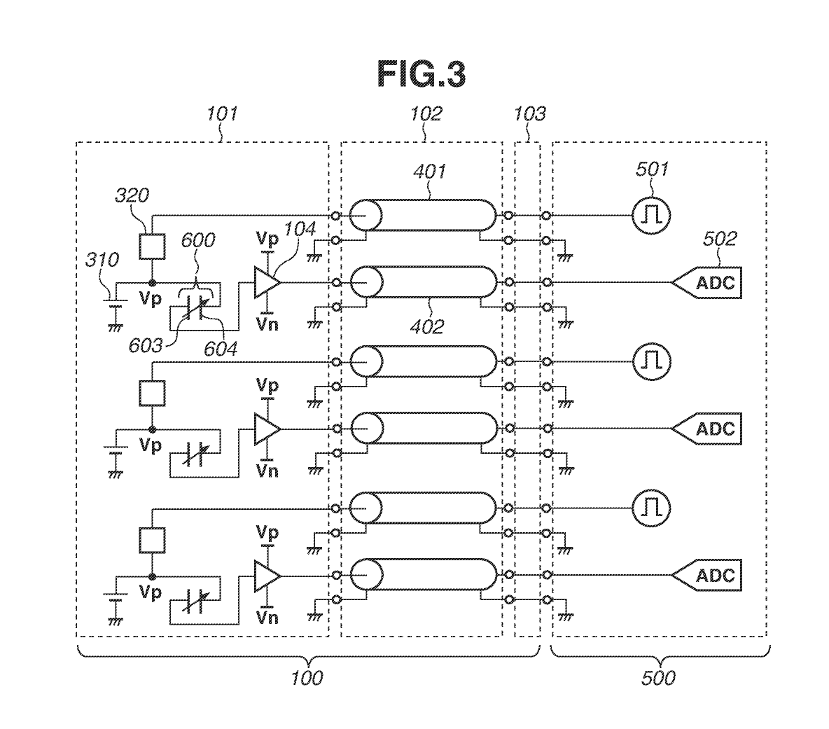

A third exemplary embodiment is different in terms of the electrode to which the preamplifier (the detection circuit) 104 is connected. The third exemplary embodiment is similar to the second exemplary embodiment except for that. The present exemplary embodiment will be described with reference to FIG. 3.

The present exemplary embodiment is characterized in that the preamplifier (the detection circuit) 104 is connected to the first electrode 603 on the oscillation membrane 602. Meanwhile, the high-voltage pulse for the transmission is applied to the second electrode 604 while being superimposed on the direct-current voltage from the direct-current voltage application unit 310.

As described above, the input terminal of the preamplifier (the detection circuit) 104 has a potential around the reference potential of the preamplifier 104 (normally, the ground potential, and a potential of several volts at most). The potential of the first electrode 603 connected to the input terminal of the preamplifier 104 is also fixed to a potential around the ground potential. On the other hand, the potential in which the high-voltage pulse for the transmission is superimposed on the direct-current voltage has a potential in a range from several dozen volts to several hundred volts. The direct-current voltage applied in the CMUT 600 is a high voltage from several dozen volts to several hundred volts, and therefore is desirable to be positioned on an inner side of the ultrasonic probe 100 as much as possible. In addition, the high-voltage pulse for the transmission is also as high as several dozen volts, and therefore is also desirable to be positioned on the inner side of the ultrasonic probe 100 (at a position away from the subject) as much as possible. In the commonly used CMUT, the direct-current voltage and the high-voltage pulse are applied to different electrodes. Thus, it is difficult to position both of them on the inner side of the probe. However, in the present exemplary embodiment, the voltage-level shift unit (the transmission signal level shift unit) 320 is included, and thus the ultrasonic probe 100 is configured to superimpose the high-voltage pulse for the transmission on the direct-current voltage. Accordingly, the high-voltage signals are collected to the same electrode. Therefore, the present exemplary embodiment allows both the direct-current voltage and the high-voltage pulse for the transmission to be positioned on the inner side of the probe 100.

Then, comparing a positional relationship between an object (a subject) targeted for ultrasonic measurement using the ultrasonic transducer according to the present exemplary embodiment and the first electrode 603 to the positional relationship with this object in the first exemplary embodiment of the present disclosure, the ultrasonic probe 100 according to the third exemplary embodiment is configured in such a manner that the electrode applying the high voltage is disposed at a position away from the measurement target, and the further lower voltage is applied to the electrode positioned close to the measurement target. The safety of the ultrasonic probe 100 thus can be further enhanced.

According to the ultrasonic probe 100 of the present exemplary embodiment, it is possible to provide the further safe ultrasonic probe including the built-in preamplifier that can acquire an excellent wideband reception characteristic.

In the present exemplary embodiment, the ultrasonic probe 100 has been described based on the configuration including the direct-current voltage application unit 310 for each of the transmission and reception elements illustrated in FIG. 2B according to the second exemplary embodiment, but the present exemplary embodiment is not limited thereto. The present exemplary embodiment can also be employed in a similar manner for, for example, the configuration including the direct-current voltage generation unit 311 in common illustrated in FIG. 2C.

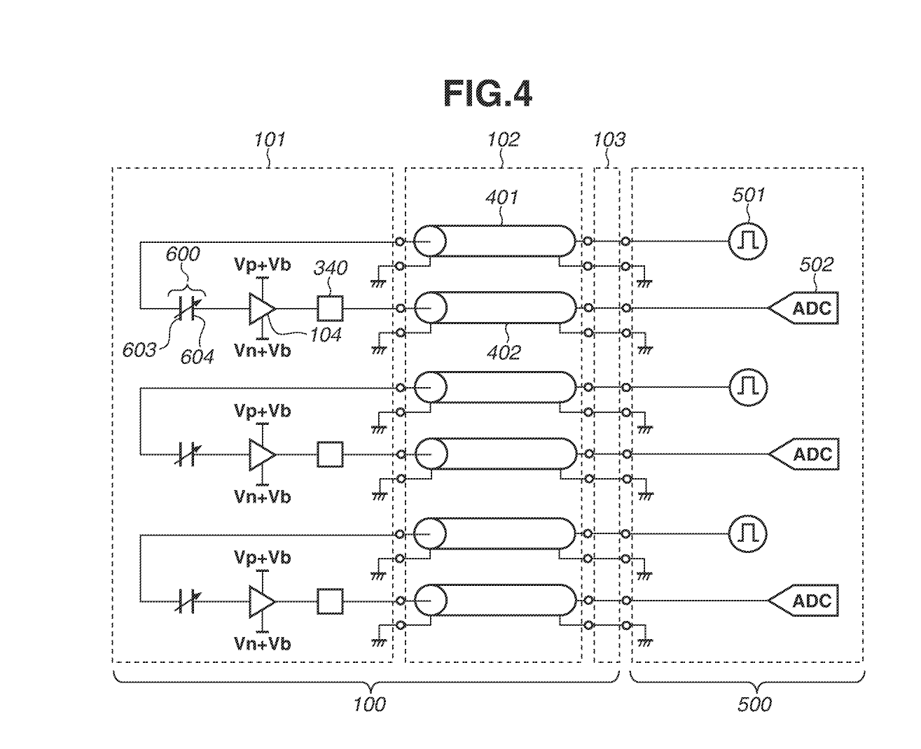

A fourth exemplary embodiment relates to the potential of the power source supplied to the preamplifier 104. The fourth exemplary embodiment is similar to any of the second to third exemplary embodiments except for that. The fourth exemplary embodiment will be described with reference to FIG. 4. FIG. 4 illustrates a voltage level shift unit 340.

In the present exemplary embodiment, the ultrasonic probe 100 is configured in such a manner that a high voltage is set as a reference Vb, for the potential of the power source supplied to the preamplifier (the detection circuit) 104 and a positive power source Vp+Vb and a negative power source Vn+Vb are applied to the preamplifier (the detection circuit) 104. This high voltage Vb is the same value as the potential difference (the bias voltage Vb) applied between the higher and lower electrodes of the CMUT 600. The potential of the input terminal of the preamplifier (the detection circuit) 104 is fixed to around the reference potential of the preamplifier 104, so that the potential of the second electrode 604 is fixed to around the high reference potential Vb in FIG. 4.

Meanwhile, the high-voltage pulse generation unit 501 in the control unit 500 generates the pulse based on the ground potential, and the potential of the high-voltage pulse generation unit 501 is fixed to the ground potential when the high-voltage pulse generation unit 501 does not generate the pulse. Therefore, according to the present exemplary embodiment, the bias voltage Vb can be applied between the higher and lower electrodes only by setting the bias voltage Vb as the reference potential of the preamplifier 104.

Further, the voltage level shift unit (a detection signal level shift unit or a reception signal level shift unit) 340 is connected to the output stage of the preamplifier 104. The voltage level shift unit (the detection signal level shift unit) 340 has a function of outputting a signal after reducing the reference potential of the signal by an amount corresponding to the bias voltage Vb when the signal passes therethrough. As a result, the preamplifier 104 operates based on the high reference potential Vb, and therefore the detection signal is output while including an offset based on the high reference potential Vb. In the present exemplary embodiment, the voltage level shift unit (the detection signal level shift unit) 340 is connected to the output stage of the preamplifier 104, and the voltage corresponding to the offset voltage Vb can be subtracted from the detection signal. Therefore, the detection signal based on the ground potential can be transmitted to the analog-digital conversion unit 502.

The voltage level shift unit (the detection signal level shift unit) 340 can be realized with use of any unit capable of converting the direct-current voltage level of the voltage (performing an operation of subtracting the bias voltage Vb) and allowing the alternating-current signal to be transmitted therethrough. For example, the voltage level shift unit 340 can be easily realized by using the capacitor as illustrated in FIG. 11A. Further, a voltage level shift circuit using a high-voltage transistor can also be used therefor.

In the configuration of the present exemplary embodiment, the reference potential of the preamplifier 104 is adjusted. Consequently, the direct-current voltage generation unit, which should be included in the CMUT in normal cases, can be omitted, so that the configuration can be simplified. Further, this configuration requires only the shift of the detection signal accompanied by a flow of a low current compared to a configuration that shifts the voltage of the high-voltage pulse accompanied by a flow of a high current, and therefore can further simplify the configuration of the voltage level shift unit 320. According to the ultrasonic probe 100 of the present exemplary embodiment, it is possible to provide the ultrasonic probe including the built-in preamplifier that has an excellent wideband reception characteristic with a simple configuration.

A fifth exemplary embodiment is similar to the first exemplary embodiment except for the configuration of the micro coaxial cables in the cable 102.

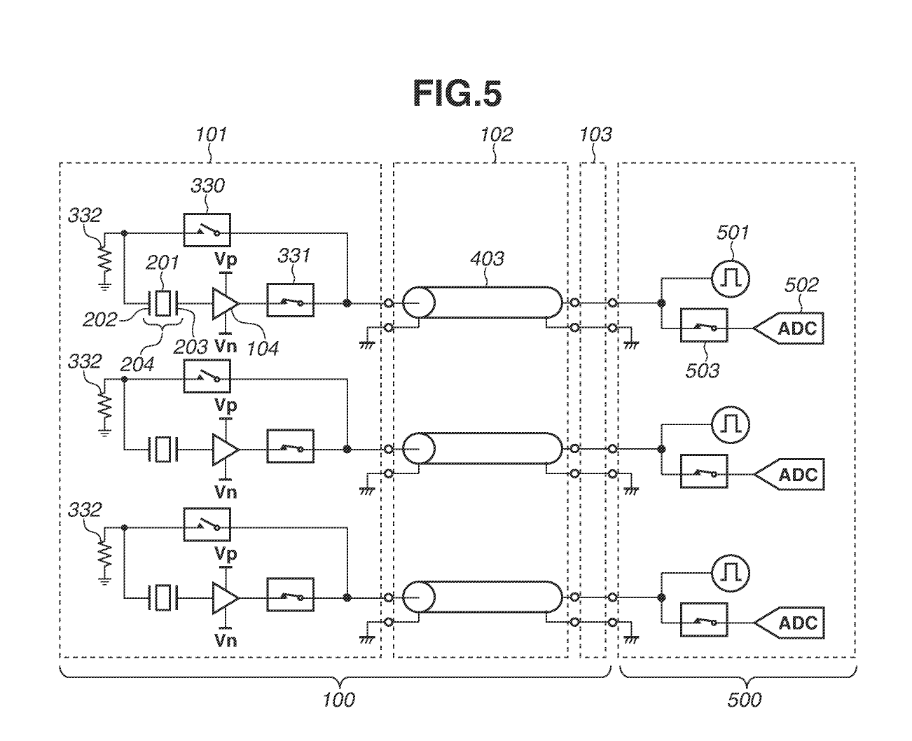

The present exemplary embodiment will be described with reference to FIG. 5.

In the above-described first to fourth exemplary embodiments, the ultrasonic probe 100 includes each of the two types of cables, the transmission cable 401 and the reception cable 402 in the cable 102 as many as the number of transmission and reception elements. In the present exemplary embodiment, a transmission and reception cable 403 serves as both the path used to transmit the high-voltage pulse to the oscillator 204 (the transmission path) and the path used to transmit the reception signal toward the control unit 500 side (the reception path) between the oscillator 204 and the control unit 500. In other words, the wiring used to transmit the transmission signal, and the wiring used to transmit the reception signal partially share the same wiring in common, and a switch configured to switch the transmission signal and the reception signal is provided in this common wiring.

First, a configuration in the control unit 500 and an operation of the control unit 500 will be described. In the control unit 500, the analog-digital conversion unit 502 is connected to the output terminal of the high-voltage pulse generation unit 501 via a control unit internal T/R switch 503, and is connected to one of ends of the transmission and reception cable 403 via the control unit internal T/R switch 503 and the connector 103. The control unit internal T/R switch 503 is a switch having a function of establishing conductivity between input and output terminals when a low voltage (normally, several volts or lower) is applied to the terminal, and blocking the conductivity between the input and output terminals when a high voltage is applied to the terminal.

As a result, at the time of the transmission, the present exemplary embodiment can prevent the high voltage from being applied to the input stage of the analog-digital conversion unit 502 when the high-voltage pulse is generated by the voltage pulse generation unit 501. Meanwhile, when the ultrasonic wave is received, the output signal from the preamplifier (the detection circuit) 104 is sufficiently lower than several volts. Therefore, the control unit internal T/R switch 503 permits the detection signal from the preamplifier 104 to be transmitted to the input stage of the analog-digital conversion unit 502 with the conductivity established between the input and output terminals. In this process, the output terminal of the voltage pulse generation unit 501 has a high impedance, and performs such an operation that the analog-digital conversion unit 502 can maintain efficiency of converting the detection signal.

Next, a configuration of the ultrasonic probe 100 according to the present exemplary embodiment will be described with reference to FIG. 5. A terminal of the transmission and reception cable 403 on an opposite side from the terminal connected to the connector 103 is connected to the first electrode 202 of the oscillator 204 via a transmission switch (a low voltage block unit) 330, and is connected to the preamplifier (the detection circuit) 104 via a T/R switch (the high voltage block unit) 331. Further, a direct-current potential fixation unit 332 is disposed at the first electrode 202 between the first electrode 202 and the ground potential. The direct-current potential fixation unit 332 can be easily constructed with use of a resistor.

The transmission switch (the low voltage block unit) 330 is a switch having a function of blocking conductivity between input and output terminals when a low voltage (normally, several volts or lower) is applied to the terminal, and establishing the conductivity between the input and output terminals when a high voltage of a predetermined value or higher is applied to the terminal.

Further, the T/R switch (the high voltage block unit) 331 is a switch having a function of establishing conductivity between input and output terminals when a low voltage of a predetermined value or lower (normally, several volts or lower) is applied to the terminal, and blocking the conductivity between the input and output terminals when a high voltage is applied to the terminal.

When the high-voltage pulse for the transmission is applied from the control unit 500 via the transmission and reception cable 403, the T/R switch (the high voltage block unit) 331 blocks the conductivity so as to prevent the high voltage from being applied to the output terminal of the preamplifier (the detection circuit) 104 and thus prevent the preamplifier 104 from being broken. Meanwhile, the other transmission switch (the low voltage block unit) 330 connected to the transmission and reception cable 403 establishes the conductivity so as to cause the high-voltage pulse to be applied to the first electrode 202 of the oscillator 204. Then, the direct-current potential fixation unit 332 is set so as to have a higher impedance value than an impedance between the electrodes of the ultrasonic probe 100. Therefore, the energy of the high-voltage pulse applied to the first electrode 202 is transmitted to the ultrasonic probe 100 side by a larger amount compared to the direct-current potential fixation unit 332, so that the ultrasonic wave can be efficiently transmitted.

On the other hand, at the time of the reception, the signal from the second electrode 203 is amplified by the preamplifier (the detection circuit) 104, and is transmitted to the control unit 500 via the transmission and reception cable 403 after passing through the T/R switch (the high voltage block unit) 331 as the detection signal. In this process, the transmission switch (the low voltage block unit) 330 blocks the conductivity, so that the detection signal is transmitted to the control unit 500 side without being degraded. The present exemplary embodiment is characterized in that the direct-current potential fixation unit 332 is connected to the first electrode 202. By this configuration, the potential of the first electrode 202 is fixed at the ground potential when a transmission switch (a low voltage block unit) 330 blocks the conductivity. If the potential of the first electrode 202 is not fixed but is floating, a failure may occur in the operation of detecting the reception signal. The present exemplary embodiment can prevent or reduce this failure, and perform a stabile reception operation.

The transmission switch (the low voltage block unit) 330 can be easily realized by a configuration in which two diodes are connected while being oriented in opposite directions from each other as illustrated in FIG. 11C. The present exemplary embodiment is not limited to the configuration illustrated in FIG. 11C, and can be realized in a similar manner with use of any configuration having the function of blocking the conductivity between the input and output terminals, and establishing the conductivity between the input and output terminals when the high voltage is applied to the terminal. Desirably, a lowermost voltage VL that the transmission switch (the low voltage block unit) 330 permits to be transmitted therethrough is sufficiently low compared to the voltage of the high-voltage pulse so as to prevent degradation of the high-voltage pulse signal to be transmitted. Especially, setting 1.0 volt or lower as the lowermost voltage VL to be transmitted can keep degradation of energy to be introduced into the ultrasonic transducer at 1 percent or lower when a high-voltage pulse of 100 volts is applied.

A forward voltage drop of the diode made from silicon that is used in the circuit illustrated in FIG. 11C is from 0.6 volts to 0.7 volts. The circuit illustrated in FIG. 11C therefore allows a sufficient reduction in the lowermost voltage VL to be transmitted, and can operate so as to prevent or reduce the degradation when the high-voltage pulse is transmitted.

The T/R switch (the high voltage block unit) 331 can be easily realized by a bridge configuration using a diode as illustrated in FIG. 11D. The present exemplary embodiment is not limited to the configuration illustrated in FIG. 11D. The present exemplary embodiment can be realized in a similar manner with use of any configuration having the function of establishing the conductivity between the input and output terminals when the low voltage (normally, several volts or lower) is applied to the terminal, and blocking the conductivity between the input and output terminals when the high voltage is applied to the terminal. For example, as another possible configuration, the present exemplary embodiment can be realized with use of, for example, a switch that detects an applied voltage to perform the operation of establishing and blocking the conductivity with use of a transistor. Further, an uppermost voltage VH that the T/R switch (the high voltage block unit) 331 permits to be transmitted therethrough should be an amplitude of the detection signal or larger to correctly transmit the amplitude of the detection signal. Therefore, any voltage that is the amplitude of the detection signal or larger under use conditions can be used for the uppermost voltage VH to be transmitted. Generally, the magnitude of the detection signal acquired by amplifying the reception signal at the ultrasonic transducer is 0.5 volts at most. Therefore, setting 0.5 volts or higher as the uppermost voltage VH that the T/R switch (the high voltage block unit) 331 permits to be transmitted therethrough allows the reception signal to be transmitted to the control unit 500 side without being clipped even with respect to various types of ultrasonic transducers.

In the present exemplary embodiment, the transmission and reception cable 403 used to transmit the transmission signal and the reception signal branches into the path for transmission (the transmission path) and the path for the reception (the reception path) in the ultrasonic probe casing 101. The transmission path side is connected to the first electrode 202 via the transmission switch (the low voltage block unit) 330 that prohibits the low voltage from being transmitted and permits the high voltage of the predetermined voltage value or higher to be transmitted therethrough. On the other hand, the reception path side is connected to the second electrode 203 via the T/R switch (the high voltage block unit) 331 that prohibits the high voltage from being transmitted and permits only the low voltage to be transmitted therethrough and the preamplifier 104 beyond that. Due to this arrangement, the ultrasonic probe 100 may be configured to use the transmission and reception cable 403 serving as both the cable used to apply the transmission pulse from the control unit 500 side (the cable 401 in the previous exemplary embodiments) and the cable used to transmit the reception signal to the control unit 500 side (the cable 402 in the previous exemplary embodiments). Even such a configuration also eliminates the necessity of adding the protection circuit to the input terminal of the preamplifier 104, and therefore can acquire a reception signal with an excellent reception characteristic. The configuration illustrated in FIG. 5 includes the T/R switch 331. Therefore, when the high-voltage pulse is applied to the transmission and reception cable 403 used for both the transmission and the reception at the time of the transmission of the ultrasonic wave, the path of the wiring connected to the preamplifier 104 is closed off, and the high voltage is prevented from being applied to the output terminal of the preamplifier 104. Further, at the time of the reception of the ultrasonic wave, because the output signal output from the preamplifier 104 is approximately several hundred microvolts, the T/R switch 331 does not block the conductivity and the reception signal is transmitted to the signal processing unit 500 without being blocked. The T/R switch 331 is connected to the output terminal side of the preamplifier 104, so that the configuration according to the present exemplary embodiment is configured to be difficult to affect the reception characteristic of the preamplifier 104. If the T/R switch 331 is not provided, the high-voltage pulse at the time of the transmission is applied to the output terminal of the preamplifier 104, and the preamplifier 104 may be broken in the worst case. The configuration according to the present exemplary embodiment can prevent that. The configuration illustrated in FIG. 5 is the configuration in which the high voltage is not applied to the preamplifier 104, and therefore allows the same cable to be used for both the transmission and the reception as a cable for use in the transmission and the reception that is connected to the apparatus side.

Further, this configuration includes the provision of the transmission switch 330, and therefore can prevent the detection signal from being wrapped around toward the second electrode 203 side from a branch point of the transmission and reception cable 403 used for both the transmission and the reception. The detection signal is low compared to the high-voltage pulse applied at the time of the transmission of the transmission and the reception. However, if further accurate transmission of the ultrasonic wave is attempted, this attempt makes it impossible to ignore the influence of the wraparound of the detection signal. The present configuration includes the transmission switch 330 that prohibits a constant voltage from being transmitted therethrough in the path into which the detection signal will be wrapped around, and therefore can reduce the wraparound of the detection signal and transmit the ultrasonic wave with further high accuracy. Consequently, further correct transmission and reception can be achieved.

According to the ultrasonic probe 100 of the present exemplary embodiment, it is possible to provide the ultrasonic probe including the built-in preamplifier that can reduce the number of cables required for connecting to the control unit and has an excellent reception characteristic.

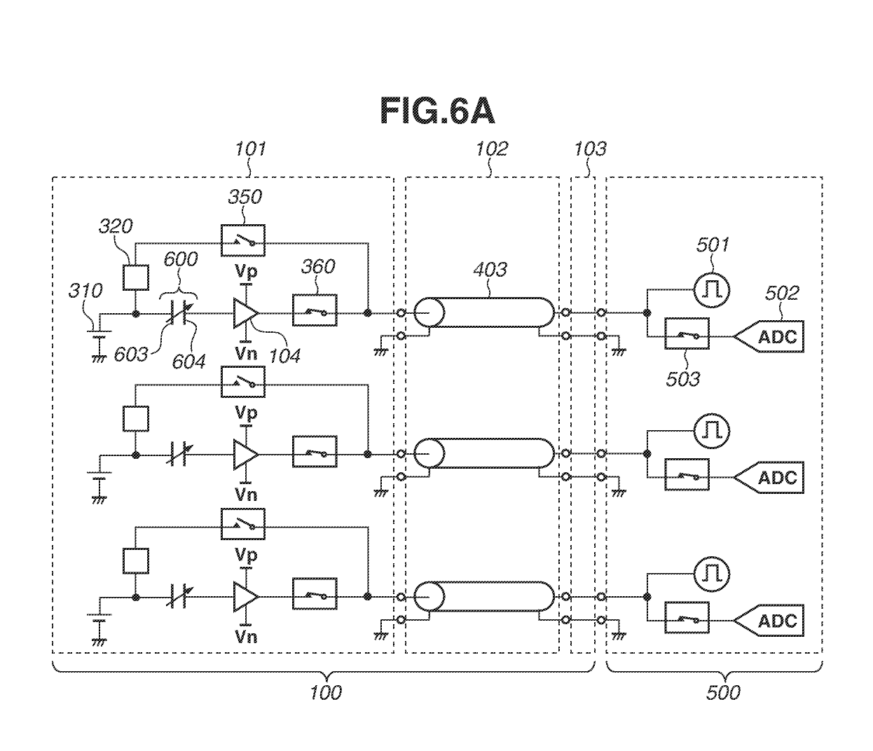

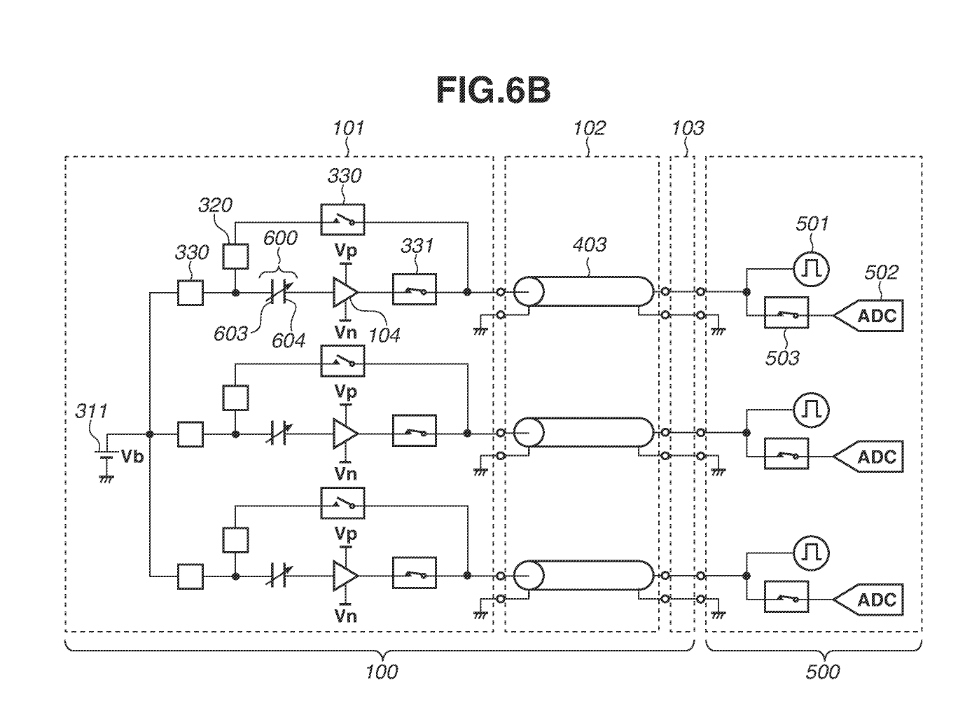

A sixth exemplary embodiment is a configuration in which the ultrasonic transducer according to the second exemplary embodiment is applied to the configuration according to the fifth exemplary embodiment. The sixth exemplary embodiment will be described with reference to FIG. 6A and 6B.

In the present exemplary embodiment, the ultrasonic probe 100 uses the transmission and reception cable 403 for exchanging the transmission and reception signals with the control unit 500. The transmission and reception cable 403 used to transmit the transmission signal and the reception signal branches into the path for the transmission (the transmission path) and the path for the reception (the reception path) in the probe casing (ultrasonic probe casing) 101. The transmission path side is connected to the first electrode 603 of the CMUT 600 via a transmission switch (the low voltage block unit) 350 and the voltage level shift unit (the transmission signal level shift unit) 320. The first electrode 603 of the CMUT 600 is connected to the direct-current potential generation unit 310 which is independently provided. On the other hand, the reception path side is connected to the second electrode 604 of the CMUT 600 via a T/R switch (the high voltage block unit) 360 and the preamplifier 104 beyond that.

In the present exemplary embodiment, the ultrasonic probe 100 includes the transmission switch (the low voltage block unit) 350 and the voltage level shift unit (the transmission signal level shift unit) 320 in the transmission path beyond the transmission and reception cable 403. This voltage level shift unit 320 included in the present exemplary embodiment allows the high-voltage pulse based on a different reference potential to match the potential of the first electrode 603. In other words, the high-voltage pulse based on the ground potential from the control unit side can be superimposed on the direct-current voltage and then applied to the first electrode 603 of the CMUT 600. The potential of the second electrode 604 is fixed at the reference potential of the preamplifier 104 (the ground potential in many cases), so that the potential difference including the predetermined potential difference Vb and further the high-voltage pulse superimposed thereon can be applied to between the first electrode 603 and the second electrode 604. Accordingly, even in the configuration using the same cable as both the transmission cable and the reception cable, the potential difference can be generated and the high-voltage pulse can also be applied to between the electrodes of the CMUT 600. Consequently, the transmission operation of the CMUT 600 can be stably performed.

Meanwhile, when the ultrasonic wave is received with the transmission switch (the low voltage block unit) 350 blocking the conductivity, the potential of the first electrode 603 can be defined with use of the direct-current potential generation unit 310. The potential of the second electrode 604 is fixed at the reference potential of the preamplifier 104 (the ground potential in many cases), so that the predetermined potential difference Vb can be applied to between the first electrode 603 and the second electrode 604. Accordingly, the potential difference can be generated between the electrodes of the CMUT 600. Consequently, the reception operation of the CMUT 600 can be stably performed. Further, the CMUT 600 is used as the ultrasonic transducer, so that the ultrasonic probe 100 can detect a wideband reception signal with an excellent reception characteristic.

According to the ultrasonic probe 100 of the present exemplary embodiment, it is possible to provide the ultrasonic probe including the built-in preamplifier that has the reduced number of cables required for connecting to the control unit and has a wideband excellent reception characteristic.

In the present exemplary embodiment, the ultrasonic probe 100 has been described with use of the direct-current potential generation unit 310 which is independently provided, but the present exemplary embodiment is not limited thereto. A configuration in which the configuration illustrated in FIG. 2C is combined with the configuration according to the fifth exemplary configuration can also be used in a similar manner (refer to FIG. 6B). In FIG. 6B, the alternating-current voltage separation unit 330 included in the present exemplary embodiment allows the ultrasonic probe 100 to manage the plurality of CMUTs 600 with use of only one direct-current potential generation unit 311. The direct-current potential generation unit should generate a high voltage and therefore should be constructed with use of a component highly resistant to a voltage. This likely leads to a large circuit configuration. The present configuration requires only one direct-current potential generation unit. Consequently, a further compact probe can be achieved. Due to this configuration, it is possible to provide the ultrasonic probe including the built-in preamplifier that has the reduced number of cables required for connecting to the control unit, is compact because requiring only one direct-current voltage generation unit, and has an excellent wideband reception characteristic. The circuit configuration has been described as being configured to include the direct-current potential generation unit in the ultrasonic probe 100 in FIGS. 2A to 2C, 3, 6A and 6B, and the like, but the present invention is not limited to this configuration. The exemplary embodiments of the present invention can also be used in a similar manner even for a configuration that includes the direct-current potential generation unit in the control unit 500 and supplies the direct-current potential to the ultrasonic probe 100 via a cable. Especially, the configurations illustrated in FIGS. 2C and 6B can operate with use of only one cable as a cable for supplying the direct-current potential, and therefore can use a further thin and flexible cable. Thus, an ultrasonic probe subject to fewer constraints in use. Especially, since the cable for supplying the direct-current potential transmits a high voltage therethrough, the cable tends to be thick. The configurations illustrated in FIGS. 2C and 6B can operate with use of only one cable as the cable for supplying the direct-current potential. Consequently, an especially significant effect can be acquired.

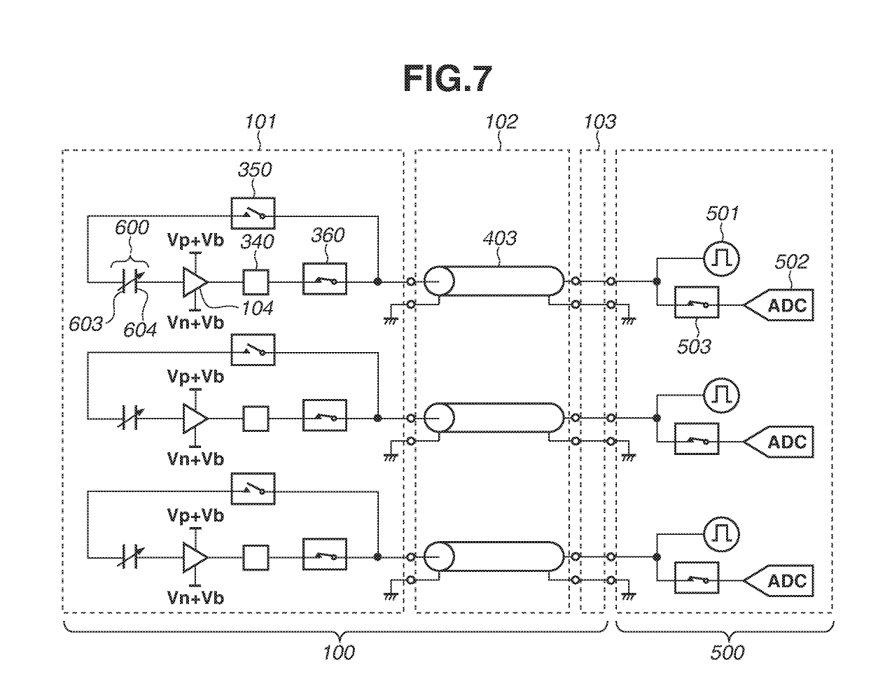

A seventh exemplary embodiment is a configuration in which the ultrasonic transducer according to the fourth exemplary embodiment is applied to the configuration according to the fifth exemplary embodiment. The seventh exemplary embodiment will be described with reference to FIG. 7.

In the present exemplary embodiment, the ultrasonic probe 100 uses the transmission and reception cable 403 for exchanging the transmission and reception signals with the control unit 500. The transmission and reception cable 403 used to transmit the transmission signal and the reception signal branches into the path for the transmission (the transmission path) and the path for the reception (the reception path) in the ultrasonic probe casing 101. The transmission path side is connected to the first electrode 603 of the CMUT 600 via the transmission switch (the low voltage block unit) 350. On the other hand, the reception path side is connected to the second electrode 604 of the CMUT 600 via the T/R switch (the high voltage block unit) 360, the voltage level shift unit (the detection signal level shift unit) 340, and the preamplifier 104 beyond that. Further, the direct-current potential fixation unit 332 is disposed at the first electrode 603 between the first electrode 603 and the ground potential. The direct-current potential fixation unit 332 can be easily constructed with use of the resistor.

In the present exemplary embodiment, the reference potential of the power source voltage applied to the preamplifier 104 is set based on the bias voltage Vb, and Vp+Vb and Vn+Vb are applied. Consequently, the bias voltage Vb can be applied between the higher and lower electrodes even with no direct-current potential application unit provided. Then, the direct-current potential fixation unit 332 is set so as to have the higher impedance value than the impedance between the electrodes of the probe 204. Therefore, when the ultrasonic wave is transmitted, the energy of the high-voltage pulse applied to the first electrode 603 is transmitted to the CMUT 600 side by a larger amount compared to the direct-current potential fixation unit 332, so that the ultrasonic wave can be efficiently transmitted. Further, since the direct-current potential fixation unit 332 is connected to the first electrode 603, the potential of the first electrode 603 is fixed at the ground potential when the transmission switch (the low voltage block unit) 350 blocks the conductivity at the time of the reception. If the potential of the first electrode 603 is not fixed but is floating, a failure may occur in the operation of detecting the reception signal. The present exemplary embodiment can prevent this failure, and perform a stabile reception operation. Further, the output signal received by the preamplifier 104 can be transmitted to the control unit 500 side after the offset corresponding to the bias voltage Vb is subtracted from this output signal by the voltage level shift unit (the detection signal level shift unit) 340. The detection signal can be therefore converted by the analog-digital conversion unit 502 in the control unit 500. Furthermore, the CMUT 600 is used as the ultrasonic transducer, so that the ultrasonic probe 100 can detect a wideband reception signal with an excellent reception characteristic. Further, the CMUT 600 does not have to include the direct-current potential generation unit, which would be necessary for the CMUT in normal cases, and the preamplifier 104 also serves as this function. Accordingly, the ultrasonic probe 100 can omit the direct-current potential generation unit using the circuit component highly resistant to a voltage. Consequently, a further size reduction can be achieved.

According to the ultrasonic probe 100 of the present exemplary embodiment, it is possible to provide the ultrasonic probe including the built-in preamplifier that has the reduced number of cables required for connecting to the control unit, has a simple configuration, and has an excellent wideband reception characteristic.

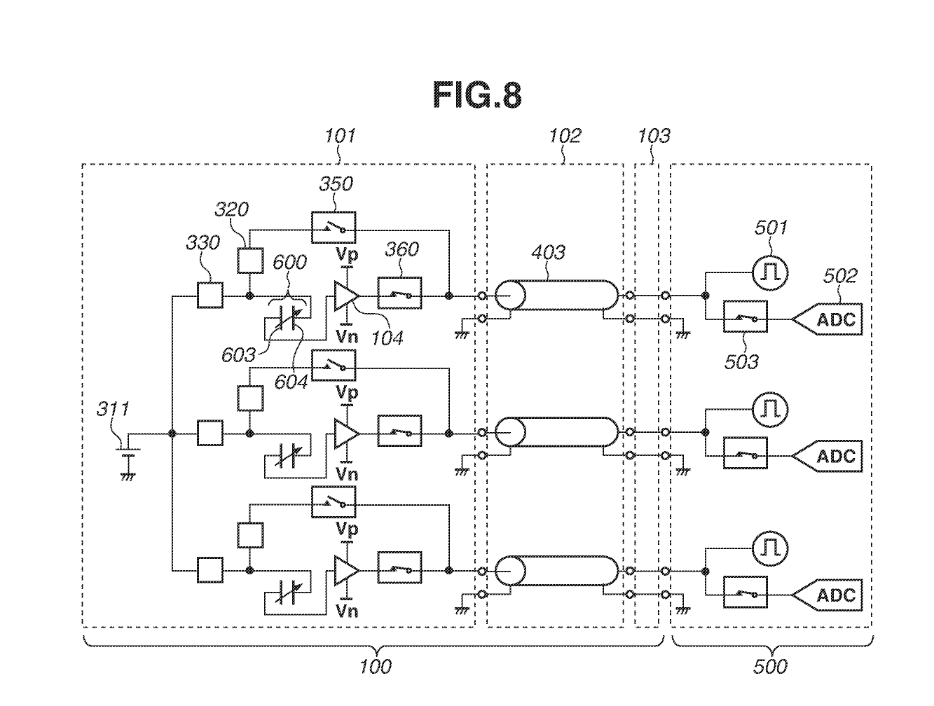

An eighth exemplary embodiment is a configuration in which the ultrasonic transducer according to the third exemplary embodiment is applied to the configuration according to the fifth exemplary embodiment. The eighth exemplary embodiment will be described with reference to FIG. 8.

In the present exemplary embodiment, the ultrasonic probe 100 uses the transmission and reception cable 403 for exchanging the transmission and reception signals with the control unit 500. The cable 403 used to transmit the transmission signal and the reception signal branches into the path for the transmission (the transmission path) and the path for the reception (the reception path) in the ultrasonic probe casing 101. The transmission path side is connected to the second electrode 604 of the CMUT 600 via the transmission switch (the low voltage block unit) 350 and the voltage level shift unit (the transmission signal level shift unit) 320. The second electrode 604 of the CMUT 600 is connected to the direct-current voltage generation unit 311 in common via the alternating-current voltage separation unit 330. The voltage level shift unit 320 included in the present exemplary embodiment allows the high-voltage pulse based on a different reference potential to match the potential of the second electrode 604. In other words, the high-voltage pulse based on the ground potential from the control unit side can be superimposed on the direct-current voltage and then applied to the second electrode 604 of the CMUT 600. The potential of the first electrode 603 is fixed at the reference potential of the preamplifier 104 (the ground potential in many cases), so that the potential difference including the predetermined potential difference Vb and further the high-voltage pulse superimposed thereon can be applied to between the first electrode 603 and the second electrode 604. Accordingly, even in the configuration using the same cable as both the transmission cable and the reception cable, the potential difference can be generated and the high-voltage pulse can also be applied to between the electrodes of the CMUT 600. Consequently, the transmission operation of the CMUT 600 can be stably performed. Meanwhile, the reception path side is connected to the first electrode 603 of the CMUT 600 via the T/R switch (the high voltage block unit) 360 and the preamplifier 104 beyond that. When the ultrasonic wave is received with the transmission switch (the low voltage block unit) 350 blocking the conductivity, the potential of the first electrode 603 can be defined with use of the direct-current potential generation unit 310. Since the potential of the second electrode 604 is fixed at the reference potential of the preamplifier 104 (the ground potential in many cases), the predetermined potential difference Vb can be applied to between the first electrode 603 and the second electrode 604. Accordingly, the potential difference can be generated between the electrodes of the CMUT 600. Consequently, the reception operation of the CMUT 600 can be stably performed.

In the present exemplary embodiment, the connection from the direct-current voltage generation unit 311 in common to the second electrode 604 of each of the transmission and reception elements is established via the alternating-current voltage separation unit 330, so that the ultrasonic probe 100 allows the ultrasonic wave to be transmitted while being separated element by element without causing a crosstalk of the high-voltage pulse applied to each of the elements. Further, the electrode to which the high voltage (the high-voltage pulse and the direct-current voltage Vb) is applied is the second electrode 604 disposed inside as viewed from the measurement target, so that the ultrasonic probe 100 can acquire further higher safety. The CMUT 600 is used as the ultrasonic transducer, so that the ultrasonic probe 100 can detect a wideband reception signal with an excellent reception characteristic.

According to the ultrasonic probe 100 of the present exemplary embodiment, it is possible to provide the ultrasonic probe including the built-in preamplifier that has the reduced number of cables required for connecting to the control unit, has higher safety, and has an excellent wideband reception characteristic.

In the present disclosure, the direct-current voltage (the bias voltage) applied to the capacitive transducer (CMUT 600) has been described as being the positive voltage relative to the ground potential, but the present exemplary embodiment is not limited to this configuration. The present exemplary embodiment can also be used in a similar manner for such a configuration that the bias voltage is a negative potential relative to the ground potential. In this case, the present exemplary embodiment can be realized by configuring the direct-current potential generation unit (the direct-current voltage application unit) 310 or the common direct-current voltage generation unit 311 to perform an appropriate operation according to the polarity of the bias voltage. Further, the present exemplary embodiment can be realized by also configuring the voltage level shift unit (the transmission signal level shift unit) 320 or the voltage level shift unit (the detection signal level shift unit) 340 to perform an appropriate operation according to the polarity of the bias voltage.

The ultrasonic probe 100 described in the description of any of the first to eighth exemplary embodiments can be used to transmit and receive ultrasonic imaging, and can be applied to a subject information acquisition apparatus including that.

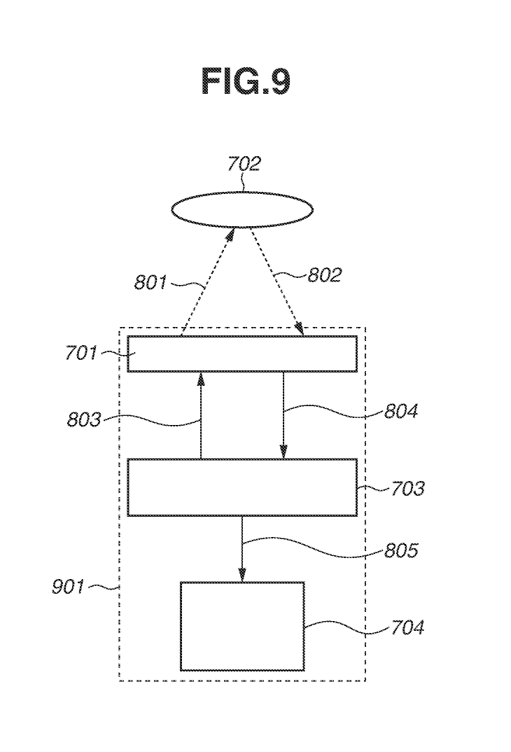

FIG. 9 is a schematic view illustrating a subject information acquisition apparatus according to a ninth exemplary embodiment. FIG. 9 illustrates an ultrasonic probe 701, a measurement target 702, an image information generation device 703, an image display device 704, a transmitted ultrasonic wave 801, and a reflected ultrasonic wave 802. Further, FIG. 9 illustrates an ultrasonic wave reception signal 803, ultrasonic wave transmission information 804, reconstructed image information 805 based on the transmission and reception signals of the ultrasonic wave, and an ultrasonic imaging system 901.

The ultrasonic wave 801 is output (transmitted) from the ultrasonic probe 701 toward the measurement target 702. The ultrasonic wave 801 is reflected on a surface of the measurement target 702 due to a difference of a characteristic acoustic impedance on this interface. The reflected ultrasonic wave 802 is received by the ultrasonic probe 701, and information of a magnitude, a shape, and a time of the reception signal is transmitted to the image information generation device 703 as the ultrasonic wave reception signal 803. On the other hand, information of a magnitude, a shape, and a time of the transmitted ultrasonic wave 801 is transmitted to the ultrasonic probe 701 as the ultrasonic transmission information 804, and, at the same time, is recorded in the image information generation device 703 as the ultrasonic wave transmission information 804. The image information generation device 703 generates an image signal of the measurement target 702 based on the ultrasonic wave reception signal 803 and the ultrasonic wave transmission information 804, and outputs the generated image signal as the reconstructed image information 805.

According to the ultrasonic probe 701 of the present exemplary embodiment, it is possible to realize the ultrasonic probe including the built-in preamplifier that has an excellent reception characteristic, so that the subject information acquisition apparatus using this ultrasonic probe 701 can acquire a further correct ultrasonic imaging image of the subject.

There is a measurement system (a photoacoustic imaging system) that illuminates the subject with light to cause an acoustic wave (the ultrasonic wave) to be generated from the measurement target in the subject using a photoacoustic effect, and receives the generated acoustic wave with use of an ultrasonic probe having an array configuration. The ultrasonic probe 100 according to the above-described exemplary embodiments of the present disclosure can also be used for this photoacoustic imaging system.

A tenth exemplary embodiment will be described as a configuration in which the ultrasonic probe according to the above-described exemplary embodiments of the present disclosure is applied to a subject information acquisition apparatus that carries out the photoacoustic imaging in addition to the ultrasonic imaging described in the description of the ninth exemplary embodiment.

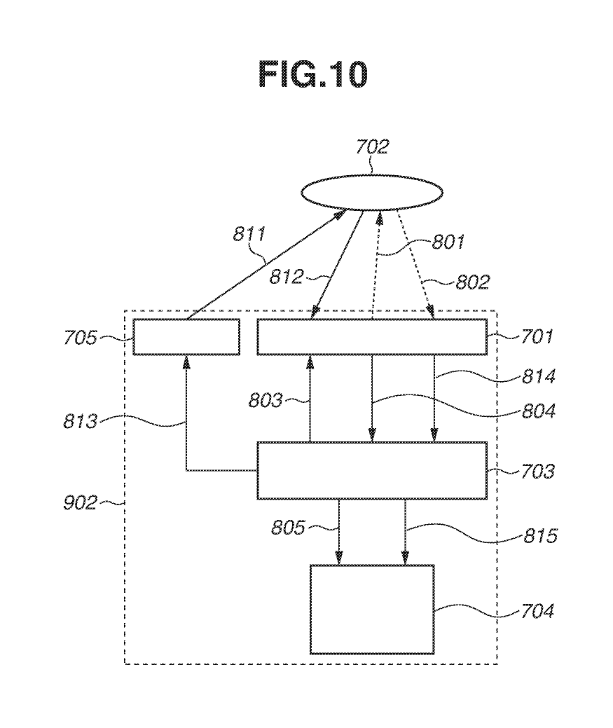

FIG. 10 is a schematic view illustrating a subject information acquisition apparatus according to the present exemplary embodiment. FIG. 10 illustrates a light source 705, light 811, an acoustic wave (an ultrasonic wave) 812 generated from irradiation with the light 811, a photoacoustic wave reception signal 814, and a light emission instruction signal 813. Further, FIG. 10 illustrates reconstructed image information 815 based on the photoacoustic signal, and an ultrasonic imaging system 902 equipped with a photoacoustic imaging function.

In the following description, an operation in the photoacoustic imaging will be described. First, the light 811 (pulsed light) is generated from the light source 705 based on the light emission instruction signal 813, by which the measurement target 702 is irradiated with the light 811. The photoacoustic wave (the ultrasonic wave) 812 is generated in the measurement target 702 due to the irradiation with the light 811, and this ultrasonic wave 812 is received by the plurality of oscillators 204 included in the ultrasonic probe 701. Information of a magnitude, a shape, and a time of the reception signal is transmitted to the image information generation device 703 as the photoacoustic wave reception signal 814. Meanwhile, information of a magnitude, a shape, and a time (light emission information) of the light 811 generated by the light source 705 is transmitted to the light source 705 as the light emission instruction signal 813, and, at the same time, is recorded in the image information generation device 703. The image information generation device 703 generates an image signal of the object (the subject) 702 based on the photoacoustic wave reception signal 814 and the light emission information 813, and outputs the generated image signal as the reconstructed image information 815 based on the photoacoustic signal.

After that, the measurement target 702 is displayed as an image on the image display device 704 based on the two pieces of information, i.e., the reconstructed image information 815 based on the photoacoustic signal, and the reconstructed image information 805 based on the transmission and reception of the ultrasonic wave that is acquired by the method described in the description of the above-described ninth exemplary embodiment.

In this manner, the ultrasonic probe 701 according to the present exemplary embodiment can realize the ultrasonic probe including the built-in preamplifier, so that the subject information acquisition apparatus using this ultrasonic probe 701 can acquire a further correct photoacoustic imaging image of the subject.

The subject information acquisition apparatus according to the present exemplary embodiment forms an image by acquiring the reception information according to the different measurement method called the photoacoustic method in addition to the transmission and reception of the ultrasonic wave. Therefore, the subject information acquisition apparatus can acquire and display the image with a further larger information amount from the measurement target 702.

Further, in the present exemplary embodiment, the reception of the photoacoustic wave and the transmission and reception of the ultrasonic wave are carried out by one ultrasonic probe 701, so that the image can be acquired with further little misalignment in a relative position between the photoacoustic imaging image and the ultrasonic imaging image.

The present exemplary embodiment has been described as the subject information acquisition apparatus that carries out the photoacoustic imaging in addition to the ultrasonic imaging, but the present disclosure is not limited thereto. The ultrasonic probe according to the present disclosure can also be applied to a subject information acquisition apparatus that carries out only the photoacoustic imaging.

In the present disclosure, the term “acoustic wave” is intended to include elastic waves called a photoacoustic wave, a photoacoustic-ultrasonic wave, a sound wave, and an ultrasonic wave, and the acoustic wave generated due to the irradiation with the light is especially referred to as the “photoacoustic wave”. Further, among types of the acoustic wave, the acoustic wave transmitted from the probe may be referred to as the “ultrasonic wave”, and the transmitted ultrasonic wave reflected in the subject may be especially referred to as the “reflected wave”. The term “ultrasonic wave” may be used as a representative of the acoustic wave. According to the present disclosure, the ultrasonic probe including the preamplifier configured to amplify the reception signal of the ultrasonic wave can transmit and receive the ultrasonic wave without requiring the protection circuits in front of and at the back of the preamplifier. Therefore, the ultrasonic probe can eliminate the influence on the reception characteristic of the preamplifier due to the protection circuits, and, as a result thereof, can prevent or reduce deterioration of a sensitivity for receiving the ultrasonic wave at the ultrasonic probe.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2016-150108, filed Jul. 29, 2016, which is hereby incorporated by reference herein in its entirety.

What is claimed is:

1. An ultrasonic probe comprising:
an oscillator including a pair of electrodes,
wherein the ultrasonic probe is configured to transmit and receive an ultrasonic wave by an oscillation of the oscillator, and
wherein a wiring for transmitting a transmission signal for transmitting the ultrasonic wave by the oscillator is connected to one of the pair of electrodes, and a preamplifier configured to amplify a reception signal obtained from the reception of the ultrasonic wave by the oscillator is connected to the other of the pair of electrodes.

2. The ultrasonic probe according to claim 1, wherein the wiring for transmitting the transmission signal and a wiring for transmitting the reception signal form a common wiring in which a wiring is partially shared between the transmission signal and the reception signal, and a switch configured to switch a state capable of transmitting the transmission signal and a state capable of transmitting the reception signal is provided in this common wiring.

3. The ultrasonic probe according to claim 1, further comprising a piezoelectric element between the pair of electrodes,
wherein the ultrasonic probe is configured to transmit and receive the ultrasonic wave using a piezoelectric effect of the piezoelectric element.

4. The ultrasonic probe according to claim 1, further comprising a space between the pair of electrodes,
wherein the ultrasonic probe is configured to transmit and receive the ultrasonic wave by a change in a distance between the pair of electrodes that is caused by the oscillation of the oscillator.

5. The ultrasonic probe according to claim 4, wherein a potential generation unit configured to generate a direct-current potential, and a voltage level shift unit configured to shift a voltage level are connected to the one of the pair of electrodes.

6. The ultrasonic probe according to claim 1, wherein the oscillator includes a plurality of oscillators, and a potential generation unit configured to generate a direct-current potential is connected to and shared between the plurality of oscillators.

7. The ultrasonic probe according to claim 1, wherein a transmission signal level shift unit configured to shift a level of the transmission signal, and a low voltage block unit configured to establish conductivity when a voltage of a predetermined value or higher is applied are connected in this order to an electrode to which a path for applying the transmission signal is connected, and
wherein the preamplifier and a high voltage block unit configured to establish conductivity when a voltage of a predetermined value or lower is applied are connected in this order to the other of the pair of electrodes.

8. The ultrasonic probe according to claim 7, wherein the low voltage block unit does not permit transmission of a voltage of 1.0 volt or lower, and the high voltage block unit does not permit transmission of a voltage of 0.5 volts or higher.

9. The ultrasonic probe according to claim 1, wherein a reception signal level shift unit configured to shift a detection signal level of the reception signal is connected between the preamplifier and a high voltage block unit.

10. The ultrasonic probe according to claim 9, wherein a capacitive transducer includes a plurality of elements, and each of electrodes of the plurality of elements to which a path for applying the transmission signal is connected is connected to a common potential application unit via an alternating-current voltage separation unit.

11. The ultrasonic probe according to claim 1, wherein a protection circuit for protecting the preamplifier is not provided.

12. The ultrasonic probe according to claim 1, wherein a distance between a target object for measurement of the ultrasonic wave with use of an ultrasonic transducer and a first electrode is shorter than a distance between the target object and a second electrode, and the preamplifier configured to detect the reception signal is connected to the first electrode and a path for applying the transmission signal is connected to the second electrode.

13. The ultrasonic probe according to claim 1, wherein the oscillator includes a plurality of oscillators, and each of the electrodes of the plurality of oscillators is connected to a common potential application unit via an alternating-current voltage separation unit, a corresponding one of the wirings for transmitting the transmission signals of the plurality of oscillators is connected to each of the electrodes of the plurality of oscillators.

14. The ultrasonic probe according to claim 1, wherein a low voltage block unit configured to establish conductivity when a voltage of a predetermined value or higher is applied is connected to one of the pair of electrodes of the oscillator to which the wiring for transmitting the transmission signal is connected, and
wherein the preamplifier and a high voltage block unit configured to establish conductivity when a voltage of a predetermined value or lower is applied are connected in this order to the other of the pair of electrodes.

15. The ultrasonic probe according to claim 14, further comprising a transmission signal level shift unit configured to shift a level of the transmission signal to a level between the electrode of the oscillator to which the wiring for transmitting the transmission signal is connected and the low voltage block unit.

16. The ultrasonic probe according to claim 7, wherein a direct-current potential fixation unit is connected to the electrode of the oscillator that is electrically connected to the low voltage block unit.

17. An ultrasonic apparatus comprising:
the ultrasonic probe comprising:
an oscillator including a pair of electrodes,
wherein the ultrasonic probe is configured to transmit and receive an ultrasonic wave by an oscillation of the oscillator, and
wherein a wiring for transmitting a transmission signal for transmitting the ultrasonic wave by the oscillator is connected to one of the pair of electrodes, and a preamplifier configured to amplify a reception signal obtained from the reception of the ultrasonic wave by the oscillator is connected to the other of the pair of electrodes,
a voltage pulse generation unit configured to be used to generate the transmission signal; and
an analog-digital conversion unit configured to convert the reception signal from an analog signal into a digital signal.
